(19) United States
(12) Patent Application Publication
(10) Pub. No.: US 2022/0139512 A1
(43) Pub. Date: May 5, 2022

(54) MAPPING PATHOLOGY AND RADIOLOGY ENTITIES

(71) Applicant: KONINKLIJKE PHILIPS N.V., EINDHOVEN (NL)

(72) Inventors: VADIRAJ HOMBAL, WAKEFIELD, MA (US); REBECCA MIELOSZYK, BOTHELL, WA (US); SANDEEP MADHUKAR DALAL, WINCHESTER, MA (US); PRESCOTT PETER KLASSEN, CAMBRIDGE, MA (US)

(21) Appl. No.: 17/430,073

(22) PCT Filed: Feb. 11, 2020
(86) PCT No.: PCT/EP2020/053403

§ 371 Date: Aug. 11, 2021

Related U.S. Application Data
(60) Provisional application No. 62/806,058, filed on Feb. 15, 2019.

(51) Int. Cl. G16H 15/00

(52) U.S. Cl. CPC G16H 15/00

(57) ABSTRACT

Embodiments described herein relate to generating and using mappings between entities and entity features extracted from pathology and radiology reports. In various embodiments, a radiology report (102) associated with a subject (127) may be analyzed (602) to identify lesion(s); each of the lesion(s) may have lesion attribute(s). Likewise, a pathology report (112) associated with the subject may also be analyzed (604) to identify sample(s) extracted from the subject; each of the sample(s) may have sample attribute(s). Mapping(s) may be generated (606) between the one or more lesions and the one or more samples based at least in part on correlation between the lesion attributes and the sample attributes. Visual output about the radiology report and the pathology report may then be provided (608) simultaneously, and may visually emphasize a correlation between at least one of the lesions and at least one of the samples.

### FIELD OF THE INVENTION

Various embodiments described herein are directed generally to healthcare. More particularly, but not exclusively, various methods and apparatus disclosed herein relate to generating and using mappings between entities and entity features extracted from pathology and radiology reports.

### BACKGROUND OF THE INVENTION

Radiology and pathology play critical roles in patient care. While radiology is critical to identification of patient disease or malady, pathology provides the confirmatory diagnosis needed to identify the subsequent patient care path. Once a diagnosis has been confirmed, radiology and pathology are also critical to monitoring of patient progress. However, there is often a disconnect between radiology and pathology outcomes, which makes it difficult to gauge physicians' performances for evaluation, monitoring, and training purposes.

For example, a primary physician may refer a patient to a radiologist to investigate one or more symptoms the patient is experiencing. The radiologist may perform tests such as MRI, CT, X-Ray, etc., and generate a report based on his or her findings. This radiology report may be provided to the primary care physician. The primary care physician may study the report and refer the patient to a pathologist for confirmatory diagnosis. By analyzing samples taken/biopsied from the patient under a microscope, the pathologist generates a pathology report with the pathologist's findings, conclusions, etc. The pathology report is provided to the primary care physician, who can make treatment recommendations to the patient. Notably absent in this paradigm is communication or direct cooperation between the radiologist and the pathologist. In fact, if the primary care physician were to look at the radiology and pathology reports side-by-side, the primary physician would likely struggle to reconcile lesions predicted by the radiologist with samples analyzed by the pathologist to make/confirm a diagnosis.

Correlation of radiology-pathology outcomes is important to patient care. For example, radiology pathology correlation is often a topic of discussion during tumor-boards in which physicians of different specialties get together to discuss selected patient cases. It is also commonly discussed as part of educating both radiologists and pathologists. However, in these settings only a few manually selected cases are correlated. Systematic correlation of all cases is not performed because data needed to perform radiology and pathology correlation resides in separate information systems, and manual extraction of this data is labor intensive and/or cumbersome.

### SUMMARY OF THE INVENTION

The present disclosure is directed to generation and application of mappings between entities and entity features extracted from pathology and radiology reports. For example, in various embodiments, entities such as lesions may be automatically extracted from one or more radiology reports associated with a subject, e.g., using techniques such as parsing, machine learning-based sequence models, classifiers, and so forth. At the same time, entities such as samples may be automatically extracted from one or more pathology reports associated with the subject, e.g., using similar techniques. Attributes of these lesion entities and sample entities may then be analyzed to determine a mapping between individual lesions and individual samples.

Once these mappings are determined it is possible to analyze the quality of patient care between radiology and pathology on an individual patient-level and/or in the aggregate. It is also possible to generate reports and/or other visual output that emphasizes and/or annotates the relationships (e.g., correspondence) between lesions detected by a radiologist and samples analyzed by a pathologist. For example, a radiology report and a pathology report associated with a patient may be visually annotated, e.g., using words, phrases, colors, fonts, etc., to make clear to the reader (e.g., a primary care physician caring for the patient) which lesions detected by the radiologist were confirmed/refuted by the pathologist. More generally, with techniques described herein it is possible to track patient outcomes on a systematic and/or institutional level, which in turn enables establishment of institution-wide quality and performance metrics and/or evidence-based changes to practice.

Generally, in one aspect, a method implemented using one or more processors may include: analyzing a radiology report associated with a subject to identify one or more lesions, wherein each of the one or more lesions has one or more lesion attributes; analyzing a pathology report associated with the subject to identify one or more samples extracted from the subject, wherein each of the samples has one or more sample attributes; generating one or more mappings between the one or more lesions and the one or more samples based at least in part on correlation between the lesion attributes and the sample attributes; and causing one or more computing devices to generate visual output about the radiology report and the pathology report simultaneously, wherein the visual output is generated based on the one or more mappings to visually emphasize a correlation between at least one of the lesions and at least one of the samples.

In various embodiments, one or more of the lesion attributes of a given lesion of the one or more lesions may include a location within an anatomical structure at which the lesion was identified. In various versions, one or more of the sample attributes of a given sample of the one or more samples may include a location within the anatomical structure from which the sample was biopsied. In various versions, the generating may include generating a mapping of the one or more mappings based on spatial correspondence between the location within the anatomical structure at which the lesion was identified and the location within the anatomical structure from which the sample was biopsied.

In various embodiments, the correlation between the lesion attributes and the sample attributes may include a strict matching between at least one lesion attribute and at least one sample attribute. In various embodiments, the correlation between the lesion attributes and the sample attributes may include a soft matching between at least one lesion attribute and at least one sample attribute.

In various embodiments, the correlation between at least one of the lesions and at least one of the samples may be visually emphasized using a font attribute. In various embodiments, the font attribute comprises a font color.

In addition, some implementations include one or more processors of one or more computing devices, where the one or more processors are operable to execute instructions stored in associated memory, and where the instructions are configured to cause performance of any of the aforementioned methods. Some implementations also include one or more non-transitory computer readable storage media storing computer instructions executable by one or more processors to perform any of the aforementioned methods.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE DRAWINGS

In the drawings, like reference characters generally refer to the same parts throughout the different views. Also, the drawings are not necessarily to scale, emphasis instead generally being placed upon illustrating various principles of the embodiments described herein.

FIG. 1 is a block diagram schematically depicted components of an environment in which selected aspects of the present disclosure may be implemented, in accordance with various embodiments.

FIG. 2 schematically illustrates an example of how data may be processed, beginning at radiology and pathology reports and leading to various use cases, in accordance with various embodiments.

FIG. 3 depicts an example of a radiology report and pathology report depicted side-by-side using conventional techniques.

FIG. 4 depicts an example of a radiology report and pathology report depicted side-by-side, with visual annotations added using techniques described herein.

FIGS. 5A and 5B depict examples of interfaces that may be generated using techniques described herein to illustrate correlation between radiological and pathological entities.

FIG. 6 depicts an example method for practicing selected aspects of the present disclosure.

FIG. 7 depicts an example interface that may be rendered using techniques described herein for a cohort of patients, in accordance with various embodiments.

FIG. 8 depicts an example computing system architecture on which selected aspects of the present disclosure may be implemented.

### DETAILED DESCRIPTION OF EMBODIMENTS

Radiology and pathology play critical roles in patient care. However, there is often a disconnect between radiology and pathology outcomes, e.g., due to the respective data being in separate information systems, being in different formats, being generated using different nomenclature, etc. However, correlation of radiology-pathology outcomes is important to patient care. However, when it is studied, e.g., in tumor boards, only a few manually selected cases are usually correlated. Systematic correlation is not performed because, as already mentioned, data needed to perform radiology and pathology correlation resides in separate information systems, and manual extraction of this data is labor intensive and/or cumbersome. In view of the foregoing, various embodiments and implementations of the present disclosure are directed to generating and using mappings between entities and entity features extracted from pathology and radiology reports.

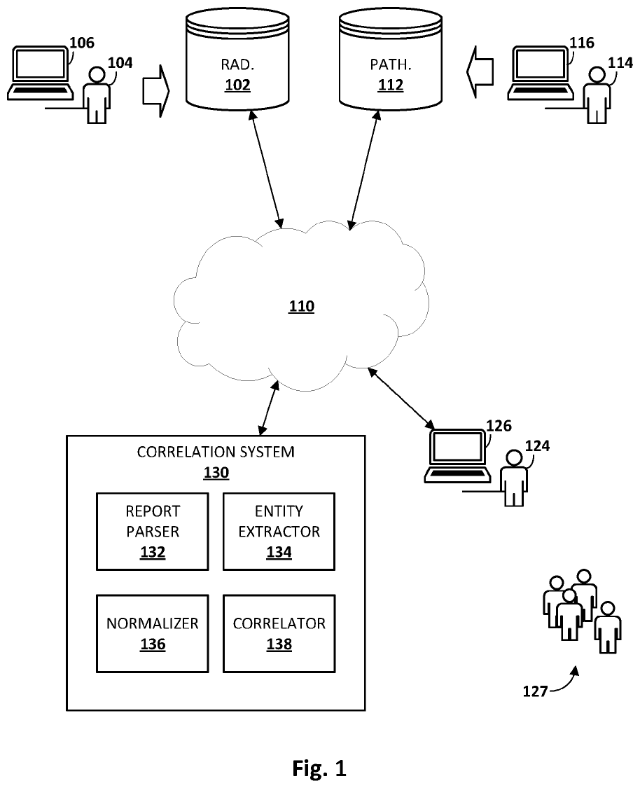

Referring to FIG. 1, an example environment is depicted that includes components such as computing devices for carrying out selected aspects of the present disclosure. The computing devices depicted in FIG. 1 may include, for example, one or more of: a desktop computing device, a laptop computing device, a tablet computing device, a mobile phone computing device, a computing device of a vehicle of the user (e.g., an in-vehicle communications system, an in-vehicle entertainment system, an in-vehicle navigation system), a standalone interactive speaker (which in some cases may include a vision sensor), a smart appliance such as a smart television (or a standard television equipped with a networked dongle with automated assistant capabilities), and/or a wearable apparatus of the user that includes a computing device (e.g., a watch of the user having a computing device, glasses of the user having a computing device, a virtual or augmented reality computing device). Additional and/or alternative client computing devices may be provided. In various embodiments, the components of FIG. 1 may be communicatively coupled with each other over one or more wired and/or wireless computer networks 110, which may include one or more local area networks and/or wide area networks such as the Internet.

As noted previously, radiology reports may be stored in a radiology information system 102 and pathology reports may be stored separately in a pathology information system 112. In FIG. 1, for example, a radiologist 104 may operate a computing device 106 to generate a radiology report that is stored in radiology information system 102. Likewise, a pathologist 114 may operate another computing device 116 to generate a pathology report that is stored in pathology information system 112. These information systems 102, 112 may take various forms, such as one or more relational databases forming part of a hospital information system, or “HIS.” Additionally or alternatively, in some cases, radiology reports and pathology reports may be stored in the same system, e.g., as part of a patient's electronic health record (“EHR”). However, even when the radiology and pathology reports are stored together, they are still often created by different people in accordance with different best practices, nomenclatures, etc.

A primary care physician 124, such as a doctor specializing in internal medicine, a pediatrician, a nurse practitioner, etc., may be a point of contact into the healthcare system for one or more patients 127. Primary care physician 124 may meet with and examine patients 127, e.g., during annual physicals and/or when patients 127 schedule appointments on demand, e.g., in response to experiencing symptoms. Based on these examinations, primary care physician 124 may refer one or more patients 127 to radiologist 104 and/or pathologist. For example, suppose test results for a given patient 127 cause the primary care physician 124 some concern. Primary care physician 124 may operate one or more computing devices 126 (or may simply pick up the telephone) to refer the given patient to radiologist 104.

Radiologist 104 may, based on findings provided by primary care physician 124, recommend various tests be performed on the given patient. These tests may include one or more of magnetic resonance imaging (“MRI”), magnetic resonance angiography (“MRA”), computerized tomography (“CT”), X-ray (e.g., mammograms), fluoroscopy (e.g., upper gastrointestinal and/or barium enemas), nuclear medicine, ultrasounds, positron emission tomography (“PET”), PET-CT, and so forth. Radiologist 104 may analyze the results of these tests (e.g., CT scans, MRIs, X-rays, etc.) to make various findings, which radiologist 104 may input into a radiology report, e.g., using computing device 106, and store in radiology information system 102. Radiologist 104 may then notify primary care physician 124 that the radiology report is ready. Primary care physician 124 may view the radiology report in various ways, such as using computing device 126.

Depending on the findings and conclusions of radiologist 104, primary care physician 124 may refer the given patient to pathologist 114 for confirmatory diagnosis. Primary care physician 124 and/or pathologist 114 may request that one or more samples by biopsied from the given patient, e.g., using needles or other surgical tools. These samples may be extracted by various intermediate medical personnel who may be selected depending on the skill level required (e.g., surgeon, technician, nurse, etc.). Once extracted the samples may or may not be further processed, e.g., by converting slices of the samples into slides that are viewable under a microscope. These slides may then be analyzed by pathologist 114, e.g., using a microscope or by scanning the slides into digital format and viewing the digital format slides on computing device 116. Based on this analysis, pathologist 114 may generate a pathology report, e.g., using computing device 116, and may store the pathology report in pathology information system 112. Primary care physician 124 may then be notified of the report. Primary care physician 124 may view the pathology report in various ways, such as using computing device 126.

In this way primary care physician 124 acts as a middleman between radiologist 104 and pathologist 114. There is no requirement that radiologist 104 and pathologist 114 confer with each other directly about the given patient, and such direct interaction between the two may in fact be rare. And there may be other entities between radiologist 104 and pathologist 114, such as technicians or other medical personnel (e.g., surgeons) that extract (biopsy) and prepare samples. Like the game of “telephone,” findings of radiologist 104—particularly abnormalities of potential concern—may be translated multiple times between the various intermediaries before reaching pathologist 114. For example, radiologist 104 may identify a lesion of concern using one type of anatomical coordinates, but the samples biopsied from the patient may be labeled (e.g., by a technician, surgeon, etc.) using another type of anatomical coordinates, or anatomical coordinates that are not as granular as those used by radiologist 104.

Consequently, when pathologist 114 creates a pathology report based on samples that were extracted in response to findings of radiologist 104, the findings, conclusions, diagnosis in the pathology report may not be readily mapped (e.g., one-to-one) with lesions identified by radiologist 104. Thus, for instance, primary care physician 124 may not be able to readily ascertain whether findings of radiologist 104 were accurate, or if radiologist 104 perhaps tends to over- or under-diagnose. Over-diagnosis is problematic for various reasons. Biopsies are invasive, may lead to complications, and should only be performed if there is a reasonably high likelihood of confirming the presence of a malady. Moreover, patients may be caused undue stress by being subjected to biopsies, regardless of the outcome. Under-diagnosis is also problematic because it may suggest that radiologist 104 is failing to adequately detect lesions or other abnormalities that he or she should detect, leading to patients not receiving timely diagnoses and/or treatment.

Accordingly, in various embodiments, a correlation system 130 may be provided to practice selected aspects of the present disclosure and address these various issues. In various implementations, correlation system 130 may include one or more computing devices communicatively coupled over one or more networks (not depicted). In some embodiments, correlation system 130 may be implemented as a “cloud-based” service. Correlation system 130 may include various modules and/or engines that may be implemented using any combination of software and hardware, including report parser 132, entity extractor 134, normalizer 136, and correlator 138. In other embodiments, one or more of 132, 134, 136, and 138 may be omitted and/or combined with other components.

Generally, correlation system 130 may be configured to generate and put to use mappings between entities and entity features extracted from pathology and radiology reports.

For example, a report parser 132 may be configured to analyze a report associated with a subject (e.g., a patient) to provide various annotations about syntactical roles played by constituent parts of sentences. In some embodiments, report parser 132 may be configured to segment radiology and/or pathology reports into sections, sections into subsections, and subsections into fundamental analytical units such as lists of key-value pairs, paragraphs, sentences, and/or individual tokens. In some embodiments, various ontology-based systems may be employed to assign sections and/or subsections to their formal or functional roles, such as “findings,” “impressions,” “diagnosis,” etc. Using ontology-based systems facilitates rapid adaptation of techniques described herein across multiple different health care systems and/or entities. In some implementations, report parser 132 may generate, as output, annotated versions of radiology and/or pathology reports.

Entity extractor 134 may be configured to analyze an annotated radiology report generated by report parser to identify one or more lesion entities (or simply, “lesions”). These one or more lesions may be identified by radiologist 104 using the various tests mentioned above (e.g., MRI, CT, etc.). Radiologist 104 may observe, and assigned to each lesion, one or more lesion attributes. Lesion attributes may include, but are not limited to, a location within an anatomical structure at which the lesion was identified, a size of the lesion, a shape of the lesion, a score assigned to the lesion by radiologist 104 (which may indicate a level of concern on the part of radiologist 104, a likelihood of cancer, etc.), a radiologist diagnosis, treatment state, and so forth. In some implementations, the lesion location attribute may be conveyed using medical coordinate standards that may or may not vary among different anatomical structures. For example, a portion of a prostate gland may be identified using a plurality of data points one or more of a “side” (e.g., right or left), a “level” (e.g., apex, mid-gland, etc.), a “zone” (e.g., peripheral, central, transition), and a “location” (e.g., posterolateral).

Similarly, entity extractor 134 may be configured to analyze a pathology report associated with a subject that has been annotated, e.g., by report parser 132, to identify one or more sample entities (or simply, “samples”). Like the lesion entities extracted from the radiology reports, these sample entities may each include various attributes that are assigned by pathologist 114 and/or by others, such as intermediate technicians that prepared the slides examined by pathologist 114. Sample attributes may include, for instance, anatomy, sample type, sample/biopsy location, sample name, sample size, a score assigned to the sample (or an abnormality within the sample), pathologist's diagnosis, histologic grade, number of cores, treatment state, and so forth. Because the differences in content between radiology and pathology reports, as well as the differences in structure and organization, in some embodiments, two distinct entity extractors 134 may be employed, one for extracting lesion entities from annotated radiology reports, and another for extracting sample entities from annotated pathology reports.

Because samples are often ordered for biopsy in response to findings by radiologist 104, in many cases there will correlation between lesions/lesion attributes extracted from the radiology report and samples/sample attributes extracted from the pathology report. This correlation may be one-to-one, many-to-one, one-to-many, or any combination thereof. For example, a single lesion spotted by radiologist 104 and conveyed in a radiology report may span multiple samples analyzed by pathologist 114. Moreover, in many circumstances physicians may operate in accordance with established “best practices” that compel them to take a predetermined series of biopsies (sometimes referred to as “blind samples”) from a plurality of predetermined locations in a particular anatomy. However, as noted previously, this correlation may not always be evident to parties such as primary care physician 124. And in some cases there may not be sufficient correlation, e.g., because a radiologist over-diagnoses or under-diagnoses, because samples are biopsied from locations that are different from the lesions identified by radiologist 104 (which may or may not indicate a sample quality issue, such as incorrect sampling, insufficient sampling, etc.), and so forth.

Entity extractor 134 may employ a variety of different techniques to extract lesion and sample entities from reports. In some implementations, entity extractor may employ machine learning models that are configured or trained to process sequences of inputs. For example, in some implementations, entity extractor 134 may employ a trained recurrent neural network which may or may not include long short-term memory network (“LSTM”) units, gated recurrent units (“GRUs”), and so forth to identify lesions and/or samples. Additionally or alternatively, in some implementations, entity extractor 134 may employ one or more conditional random fields (“CRF”) to identify lesions and/or samples.

Entity extractor 134 may also be configured to group lesions and/or samples identified using the techniques described previously with their associated attributes. Entity extractor 134 may do this in various ways. In some embodiments, entity extractor 134 may employ a text classifier such as a Maximum Entropy, or “MaxEnt,” text classifier, or another classifier such as a neural network, an ensemble tree such as an extremely randomized Tree (“ERT”), to establish lesion and sample groupings. In some embodiments, these groupings may be established in the context of sections and/or across sections.

Lesion/sample attributes may be recorded in radiology/pathology reports in various ways that reflect various nuances of individual doctors, healthcare systems, jurisdictions, regional dialects, differences in medical standards, etc. For example, the “standard of care” practiced in one jurisdiction may be different than that of another jurisdiction. Accordingly, in various embodiments, a normalizer 136 may be configured to provide an ontology-based mapping between these as-written lesion/sample attributes to “standard form attributes” that are more universally accepted and/or understood. This may facilitate downstream matching of lesions and samples. Additionally, the use of ontology-based mapping may provide for robust adaptation to differences in segmentation of various anatomies (e.g., coordinate-based segmentation of prostate and lung versus segment-based system for liver) and variation in practices across client sites (e.g., abbreviations).

Once the lesions and samples are extracted and grouped with their corresponding attributes, a correlator 138 may be configured to generate one or more mappings between the one or more lesions and the one or more samples based at least in part on correlation between the lesion attributes and the sample attributes. In some implementations, pathology samples are matched to their corresponding lesions in radiology reports and correlation is established both at a lesion-sample level and at an aggregated report level. Samples and lesions may be matched, to create a mapping between them, in various ways. In some embodiments, lesion attributes and sample attributes such as names, anatomies, and/or locations may be used to match samples to lesions, or lesions to samples.

In some embodiments, samples and lesions may be matched using “soft” or “strict” matching. With “strict” matching the lesion attributes and sample attributes may have to be virtually identical in order to create a mapping. With “soft” matching, on the other hand, some inference is permitted to map samples to lesions. For example, a sample may have location attributes that are less granular that lesions spotted by a radiologist, e.g., because the technician making the sample wasn't as specific as the radiologist. Additionally or alternatively, the sample and lesion attributes may not match perfectly because, for example, a technician took blind samples from predetermined locations that might not match exactly a granular location of a lesion identified by a radiologist. In some embodiments, soft matching may only require that there cannot be conflicts between lesion attributes and sample attributes.

Creating mappings between lesions and samples may provide a number of benefits. For example, establishing correlation at a lesion-sample level enables assessment of diagnostic discordance at a lesion level and/or assessment of biopsy yield at a sample level. These assessments can help monitor a radiologist's diagnostic accuracy and/or the quality of samples collected by intermediate parties, such as technicians, interventional radiologists, etc. Additionally or alternatively, estimating correlation across all lesions and samples pairs for a given anatomy of a given patient may enable identification of lesions-sample pairs that exhibit discordance (e.g., radiology spotted potential cancer, pathology revealed it was benign). These discordant lesion-sample pairs may be used, for instance, for special review by radiologists and pathologists either in a Tumor Board setting or elsewhere.

In various embodiments, correlation system 130 may, e.g., by way of correlator 138, cause one or more computing devices to generate visual output about the radiology report and the pathology report simultaneously. This visual output may be generated based at least in part on the one or more mappings between lesions and samples, e.g., in order to ensure that correlation(s) between at least one of the lesions and at least one of the samples is visually emphasized. For example, primary care physician 124 may operate computing device 126 to transmit a request to correlation system 130 for radiology and pathology data for a particular patient 127, e.g., prior to that patient 127 coming in for a consultation. Primary care physician 124 may then review the report(s) returned by correlation system 130 so that primary care physician 124 is able to accurately understand the relationships between lesions reported by radiologist 104 and samples analyzed by pathologist 114. This may enable primary care physician 124 to better communicate to patient 127 what primary care physician 124 believes to be the best course of action.

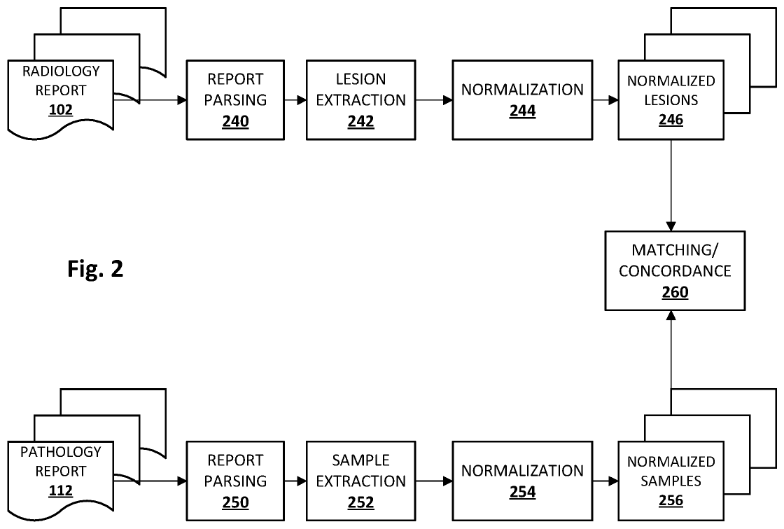

FIG. 2 schematically depicts an example process/workflow of data that may be implemented in accordance with various embodiments of the disclosure. At top is a data flow that may be implemented for extracting data from radiology reports 102. At bottom is a data flow that may be implemented for extracting data from pathology reports 112. These two data flows may be implemented in parallel, one after the other, or in any other order. Each pathology and/or radiology report may include, for instance, report data (e.g., findings, diagnoses, etc.) and metadata. Metadata may include, for instance, data identifying the patient, date of exam, accession, the exam modality employed (e.g., CT, MRI, X-ray, etc.), procedure code(s), medical record number(s) (“MRN”), body section(s) of imaging, etc. Reports 102 and 112 may be accessed, for instance, from an HIS.

In some implementations, the data process may be initiated by selection, e.g., by primary care physician 124, of a pathology report for a particular patient. This may trigger collection of past radiology reports for the same patient. These pathology and radiology reports can then be analyzed using techniques described herein. Likewise, in some implementations, the data process may be initiated by selection, e.g., by primary care physician 124, of a radiology report for a particular patient. This may trigger collection of subsequent radiology reports for the same patient.

The two data processing pipelines may operate similarly to each other. For example, at 240, report parsing of radiology report 102 may be similar to report parsing 250 of pathology report 112; operations at either of these blocks may be performed by parsing module 132, for instance, to generate annotated radiology and/or pathology reports. And parsing may include not only parsing the content of the reports, but content of the reports' metadata as well.

At block 242, one or more lesions (or lesion entities) are extracted from the annotated radiology report 102, along with one or more lesions attributes. Similarly, at block 252, one or more samples (or sample entities) are extracted from the annotated pathology report 112, along with one or more sample attributes. As mentioned previously, the operations of blocks 242 and/or 252 may be performed in various ways, such as using heuristics, machine learning models (e.g., LSTM-based recurrent neural networks), classifiers (e.g., MaxEnt, ensemble trees, etc.), or any combination thereof.

As described previously, the normalization of blocks 244 and 254 may be configured to normalize lesion and/or sample attributes into standard forms, instead of nuanced versions that may be caused by various factors, such as regional dialect, region standard of care, doctor-specific nuance, etc. Output of normalization includes normalized lesions 246 and normalized samples 256. At block 260, correlator 138 may take the normalized lesions 246 and the normalize samples 256 and correlate/match then to generate mappings between lesions and samples. As noted previously, in some embodiments in which strict matching is employed, lesions and samples may be paired by strict match of the normalized representations of their respective attributes, such as locations and/or anatomies.

With “soft” or “approximate” matching, on the other hand, lesions and samples may be matched based on partial matches of the normalized representation of their attributes, such as locations and/or anatomies. This may be anatomy specific, as all anatomies are segmented differently. In some embodiments a two pronged-strategy may be employed for soft matching. In a first prong referred to herein as “lesion dominant,” since lesions are often described in more detail than samples, all samples that partially match a lesion and do not violate any aspect of a lesion's location description are considered potential matches. Concordance may then be established in some embodiments by taking the worst outcome corresponding to the lesion. A second prong referred to herein as “sample dominant” is the inverse of “lesion dominant,” i.e. the lesion is matched to one or more candidate samples. The second prong may aide in identifying false negatives. A discordant result in a sample dominant analysis may be representative of failure to fully characterize the extent of the disease by the radiologist.

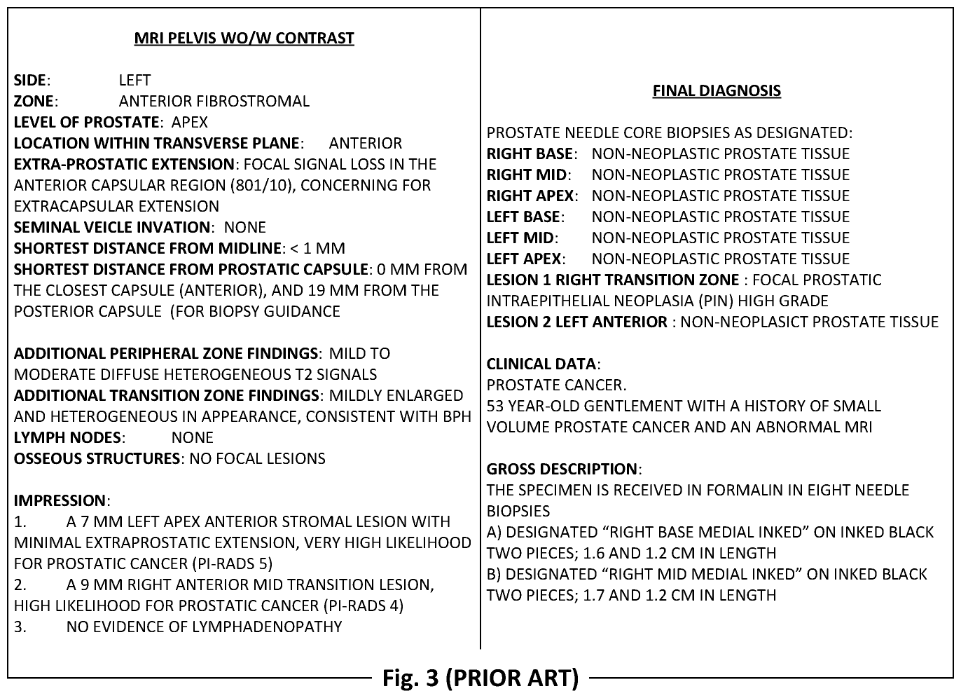

Once the lesions and samples are matched and mappings between them generated, various downstream processes may take advantage of the mappings for various purposes. FIG. 3 depicts, for a hypothetical patient, a hypothetical radiology report on the left and a hypothetical pathology report on the right. Techniques described herein have not been applied to extract lesion/sample entities from these reports. Consequently, there is no cross-report visual annotation or guidance, which may make it difficult for a primary care physician to map lesions to samples, at least in an efficiently manner and/or with much accuracy.

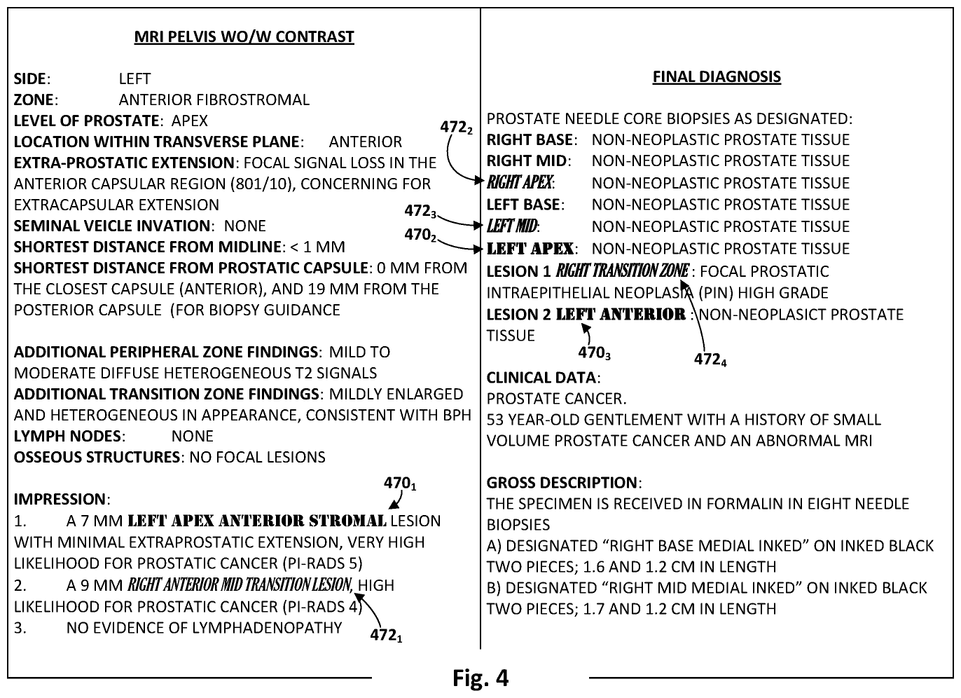

In contrast to FIG. 3, FIG. 4 depicts an example of how various segments of text in the radiology report on the left and the pathology report on the right can be visually emphasized to aid the reader in matching lesions to samples, and vice versa. While particular fonts and visual emphasis is depicted in FIG. 4, this is not meant to be limiting. Visual emphasis may be provided in any number of ways to help the reader map lesions to samples, and vice versa. For example, instead of or in addition to fonts/underlining/color, in some embodiments, arrows or other visual indicators may be rendered from lesions to matching samples, and vice versa.

In FIG. 4, a first text segment 470₁ from the radiology report has been visually emphasized using a different font than the reminder of the report. While a particular font is depicted in FIG. 4, in some embodiments, visual emphasis may instead be provided using color or another style of visual emphasis and/or annotation. A second text segment 470₂ and a third text segment 470₃ are visually emphasized in a similar manner in the pathology report at right. These visual emphasis 470₁₋₃ are meant to convey to the reader that the “7 mm left apex anterior stromal lesion” (470₁) identified under “impressions” in the radiology report is likely referring to the same area as the “Left apex: non-neoplastic prostate tissue” (470₂) and “left anterior” (470₃) in the pathology report at right.

Similarly, in the radiology report at left, the text “right anterior mid transition lesion” at 472₁ is visually emphasized the same as three segments in the pathology report: “right apex” at 472₂, “left mid” at 4723, and “right transition zone” at 472₄. Again, this suggests that all the text segments 4721-4 reference the same area, and thus represent a mapping between the lesion (472₁) and one or more samples (472₂₋₄).

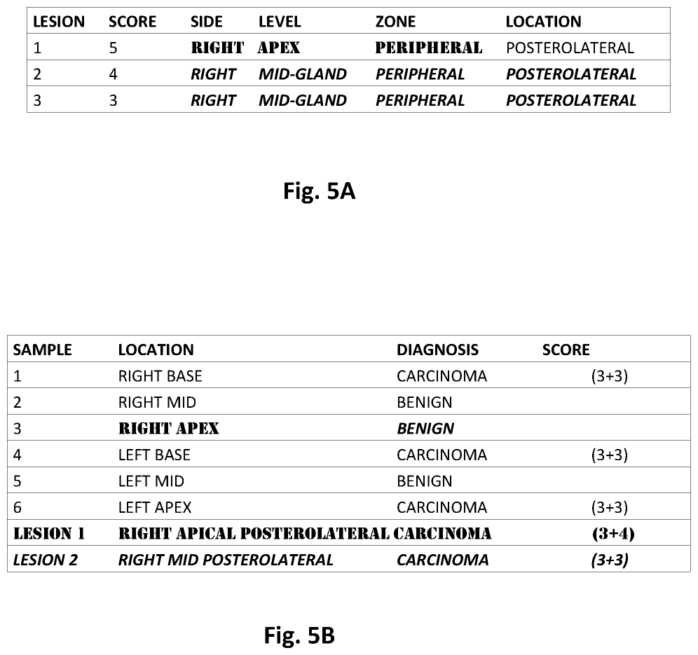

FIGS. 5A and 5B depict another example of how techniques described herein can be used to help a reader map lesions to samples, and vice versa. FIG. 5A depicts a summary report generated from a radiology report. Three lesions, 1-3, were identified by the radiologist, with lesion 1 getting a score of five (suggesting a high level of concern), lesion 2 getting a score of four (a slightly lower, but still significant, level of concern), and lesion 3 receiving a score of three (worth investigating, but not as concerning as four or five). Also identified in the table of FIG. 5A are locations at which the lesions were spotted. These locations (which are considered lesion attributes for purposes of this disclosure) are stated in terms of a coordinate scheme that may be particular to a particular anatomy, such as a prostate. Thus, for each lesion, a side, level, zone, and location is identified to aid the reader in pinpointing exactly where the radiologist spotted the lesion.

FIG. 5B depicts a summary report generated from a pathology report for the same patient as FIG. 5A. FIG. 5B identifies six blind samples (biopsies) 1-6 taken from the patient, along with two “targeted samples,” Lesion 1 and Lesion 2. Unlike the radiology report summary table of FIG. 5A, in FIG. 5B, the samples are identified with less specificity, likely because pathologist and/or other parties that help create/process samples/biopsies often do not use as much specificity. It can be seen in the table of FIG. 5B that the pathologist rated three blind samples (1, 4, 6) as containing a malady that the pathologist rated with a score of (3+3). The pathologist also rated the two targeted samples as being of concern, with Lesion 1 receiving a score of (4+3) and Lesion 2 receiving a score of (3+3).

Without visualization it may be difficult for a reader of the reports of FIGS. 5A and 5B (e.g., primary care physician 124) to match samples to lesions confidently. Accordingly, and using techniques described herein, various visual emphasis has been added to these reports to highlight which lesions map to which samples, and vice versa. For example, in FIG. 5A, lesion 1 has been visually emphasized at the text “right apex peripheral.” Correspondingly, in FIG. 5B, sample 3 and Lesion 1 have been visually emphasized with a similar font. This indicates that lesion 1 spotted by the radiologist was likely borne out in at least the Lesion 1 sample in FIG. 5B. The fact that blind sample 3 (“right apex”) turned out benign may simply suggest that the blind sampling location of sample 3 failed to yield any abnormality, but the targeted Lesion 1 did in fact yield that abnormality spotted by the radiologist as lesion 1. In a general sense this may demonstrate the effectiveness of targeted versus blind sampling.

Similarly, lesions 2 and 3 of FIG. 5A have been visually emphasized using a different visual emphasis than lesion 1. This same visual emphasis is applied to Lesion 2 in FIG. 5B to demonstrate that the targeted sample Lesion 2 corresponds to both lesions 2 and 3 in the radiology report. As noted previously, the visual emphasis depicted in FIGS. 4, 5A, and 5B is not meant to be limiting. Any type of visual emphasis, such as font color, font size, any other font attribute, animation, visual annotation, etc., may be employed to convey to the reader the relationships between lesions and samples.

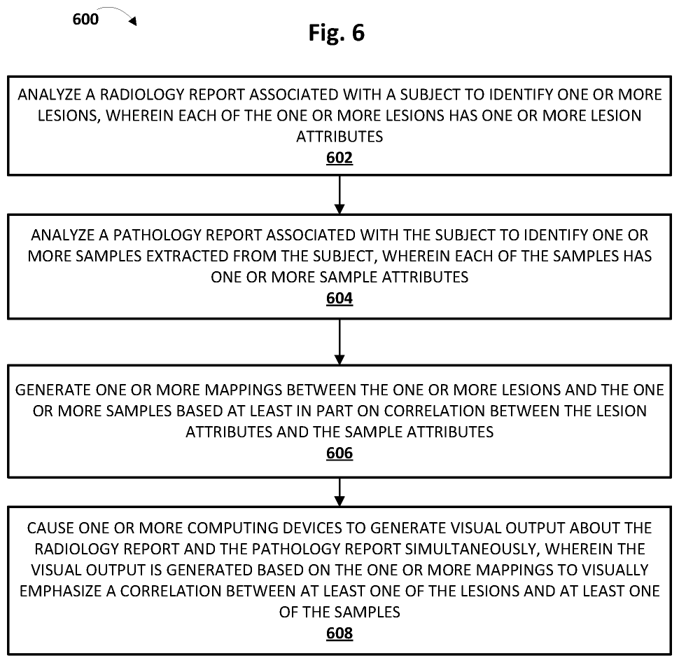

FIG. 6 illustrates a flowchart of an example method 600 for practicing selected aspects of the present disclosure. The operations of FIG. 6 can be performed by one or more processors, such as one or more processors of the various computing devices/systems described herein. For convenience, operations of method 600 will be described as being performed by a system configured with selected aspects of the present disclosure. Other implementations may include additional operations than those illustrated in FIG. 6, may perform operations (s) of FIG. 6 in a different order and/or in parallel, and/or may omit one or more of the operations of FIG. 6.

At block 602, the system may analyze a radiology report associated with a subject to identify one or more lesions. Each of the one or more lesions may have one or more lesion attributes, such as location (specified using various anatomical coordinate systems), size, shape, score, etc. As noted previously, in some embodiments the operations of block 602 may include parsing the radiology report (block 240 of FIG. 2), extracting the lesions (block 242), and normalization (block 244).

At block 604, which may be performed after, before, or concurrently with the operations of block 602, the system may analyze a pathology report associated with the subject to identify one or more samples extracted from the subject. Each of the samples may have one or more sample attributes, such as location, name, shape, size, score, diagnosis, etc. As noted previously, in some embodiments the operations of block 604 may include parsing the pathology report (block 250), extracting the samples (block 252), and normalization (block 254).

At block 606, the system, e.g., by way of correlator 138, may generate one or more mappings between the one or more lesions and the one or more samples based at least in part on correlation between the lesion attributes and the sample attributes. As noted previously, this matching may be soft or strict. At block 608, the system may cause one or more computing devices, such as computing device 126 operated by primary care physician 124, to generate visual output about the radiology report and the pathology report simultaneously. The visual output in particular may be generated based on the one or more mappings to visually emphasize a correlation between at least one of the lesions and at least one of the samples. Non-limiting examples of such this functionality are shown in FIGS. 4 and 5A-B.

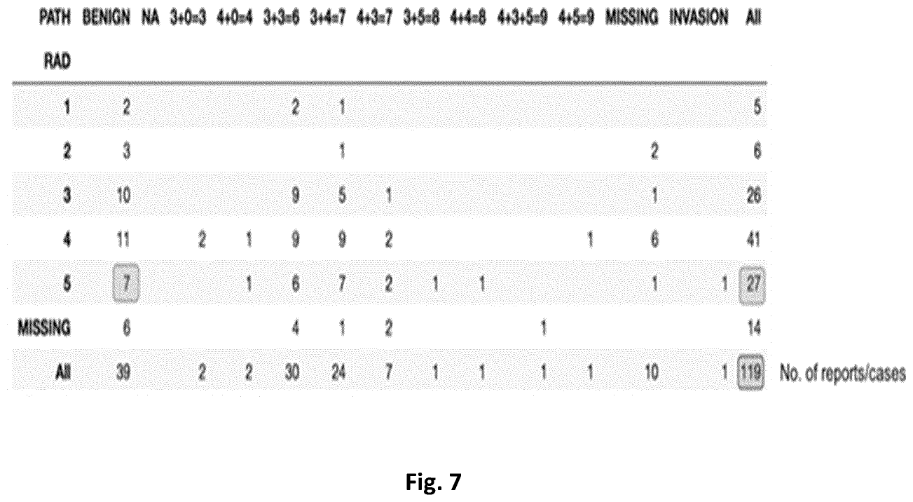

FIG. 7 depicts another example interface that may be rendered, e.g., on a display and/or in a printed report, using techniques described herein. This interface visualizes aggregate radiology and pathology concordance for a patient cohort, which allows for rapid identification and inspection of discordant cases. In this table, the very top row (“PATH”) represents the pathologist's score of lesions identified by the radiologist (Benign, NA, 3+0=3, 4+0=4, . . . , 4+5=0, Missing, Invasion, All). The left-most column represents the radiologist's scores across all the lesions. As shown at bottom right, there were a total of 119 lesions identified by the radiologist, with five being scored as “1,” six being scored as “2,” twenty-six being scored as “3,” forty-one being scored as “4,” twenty-seven being scored as “5,” and fourteen being scored as “missing.” The correlation between pathology and radiology provided by the table of FIG. 7 allows the reader to easily spot that, for instance, seven of twenty-seven patients that the radiologist identified as having a lesion with a score of five (highly concerning) actually were benign. The remaining twenty were either scored by the pathologist between 4+0=4 and 4+4=8, or placed in the missing and invasion columns. If incorrectly diagnosing lesions more than 25% of the time is deemed unacceptably high (i.e., over diagnosis), then immediate action can be taken, such as retraining the radiologist.

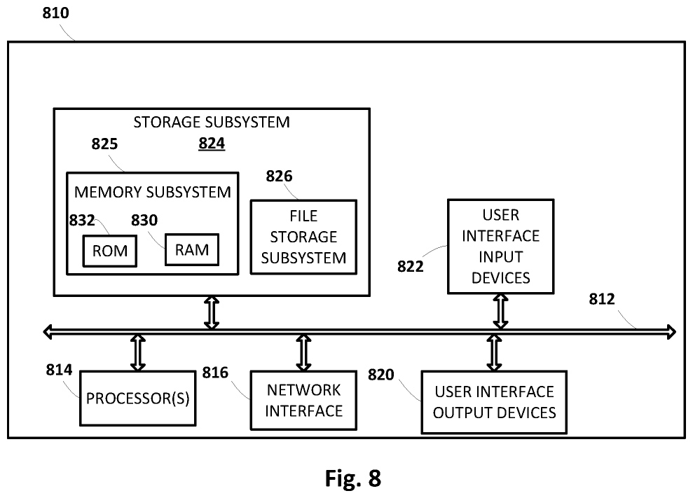

FIG. 8 is a block diagram of an example computing device 810 that may optionally be utilized to perform one or more aspects of techniques described herein. In some implementations, one or more of correlation system 130, computing devices 106, 116, 126, and/or other component(s) may comprise one or more components of the example computing device 810.

Computing device 810 typically includes at least one processor 814 which communicates with a number of peripheral devices via bus subsystem 812. These peripheral devices may include a storage subsystem 824, including, for example, a memory subsystem 825 and a file storage subsystem 826, user interface output devices 820, user interface input devices 822, and a network interface subsystem 816. The input and output devices allow user interaction with computing device 810. Network interface subsystem 816 provides an interface to outside networks and is coupled to corresponding interface devices in other computing devices.

User interface input devices 822 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computing device 810 or onto a communication network.

User interface output devices 820 may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide non-visual display such as via audio output devices. In general, use of the term “output device” is intended to include all possible types of devices and ways to output information from computing device 810 to the user or to another machine or computing device.

Storage subsystem 824 stores programming and data constructs that provide the functionality of some or all of the modules described herein. For example, the storage subsystem 824 may include the logic to perform selected aspects of the method of FIG. 6, as well as to implement various components depicted in FIGS. 1 and 2.

These software modules are generally executed by processor 814 alone or in combination with other processors. Memory 825 used in the storage subsystem 824 can include a number of memories including a main random access memory (RAM) 830 for storage of instructions and data during program execution and a read only memory (ROM) 832 in which fixed instructions are stored. A file storage subsystem 826 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 826 in the storage subsystem 824, or in other machines accessible by the processor(s) 814.

Bus subsystem 812 provides a mechanism for letting the various components and subsystems of computing device 810 communicate with each other as intended. Although bus subsystem 812 is shown schematically as a single bus, alternative implementations of the bus subsystem may use multiple busses.

Computing device 810 can be of varying types including a workstation, server, computing cluster, blade server, server farm, or any other data processing system or computing device. Due to the ever-changing nature of computers and networks, the description of computing device 810 depicted in FIG. 8 is intended only as a specific example for purposes of illustrating some implementations. Many other configurations of computing device 810 are possible having more or fewer components than the computing device depicted in FIG. 8.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be understood that certain expressions and reference signs used in the claims pursuant to Rule 6.2(b) of the Patent Cooperation Treaty (“PCT”) do not limit the scope.

1. A method implemented using one or more processors, comprising:
analyzing a radiology report associated with a subject to identify one or more lesions, wherein each of the one or more lesions has one or more lesion attributes;
analyzing a pathology report associated with the subject to identify one or more samples extracted from the subject, wherein each of the one or more samples has one or more sample attributes;
generating one or more mappings between the one or more lesions and the one or more samples based at least in part on correlation between one or more of the lesion attributes and one or more of the sample attributes; and
causing one or more computing devices to generate visual output about the radiology report and the pathology report simultaneously, wherein the visual output is generated based on the one or more mappings to visually emphasize a correlation between at least one of the lesions and at least one of the samples.

2. The method of claim 1, wherein one or more of the lesion attributes of a given lesion of the one or more lesions include a location within an anatomical structure at which the lesion was identified.

3. The method of claim 2, wherein one or more of the sample attributes of a given sample of the one or more samples include a location within the anatomical structure from which the sample was biopsied.

4. The method of claim 3, wherein the generating includes generating a mapping of the one or more mappings based on spatial correspondence between the location within the anatomical structure at which the lesion was identified and the location within the anatomical structure from which the sample was biopsied.

5. The method of claim 1, wherein the correlation between the lesion attributes and the sample attributes comprises a strict matching between at least one lesion attribute and at least one sample attribute.

6. The method of claim 1, wherein the correlation between the lesion attributes and the sample attributes comprises a soft matching between at least one lesion attribute and at least one sample attribute.

7. The method of claim 1, wherein the correlation between at least one of the lesions and at least one of the samples is visually emphasized using a font attribute.

8. The method of claim 7, wherein the font attribute comprises a font color.

9. A system comprising one or more processors and memory storing instructions that, in response to execution of the instructions by the one or more processors, cause the one or more processors to perform the following operations:
analyzing a radiology report associated with a subject to identify one or more lesions, wherein each of the one or more lesions has one or more lesion attributes;
analyzing a pathology report associated with the subject to identify one or more samples extracted from the subject, wherein each of the one or more samples has one or more sample attributes;
generating one or more mappings between the one or more lesions and the one or more samples based at least in part on correlation between one or more of the lesion attributes and one or more of the sample attributes; and
causing one or more computing devices to generate visual output about the radiology report and the pathology report simultaneously, wherein the visual output is generated based on the one or more mappings to visually emphasize a correlation between at least one of the lesions and at least one of the samples.

10. The system of claim 9, wherein one or more of the lesion attributes of a given lesion of the one or more lesions include a location within an anatomical structure at which the lesion was identified.

11. The system of claim 10, wherein one or more of the sample attributes of a given sample of the one or more samples include a location within the anatomical structure from which the given sample was biopsied.

12. The system of claim 11, wherein the generating includes generating a mapping of the one or more mappings based on spatial correspondence between the location within the anatomical structure at which one or more of the lesion was identified and the location within the anatomical structure from which the given sample was biopsied.

13. The system of claim 9, wherein the correlation between the lesion attributes and the sample attributes comprises a strict matching between at least one lesion attribute and at least one sample attribute.

14. The system of claim 9, wherein the correlation between the lesion attributes and the sample attributes comprises a soft matching between at least one lesion attribute and at least one sample attribute.

15. The system of claim 9, wherein the correlation between at least one of the lesions and at least one of the samples is visually emphasized using a font attribute.

16. The system of claim 15, wherein the font attribute comprises a font color.

17. At least one non-transitory computer-readable medium comprising instructions that, in response to execution of the instructions by one or more processors, cause the one or more processors to perform the following operations:
analyzing a radiology report associated with a subject to identify one or more lesions, wherein each of the one or more lesions has one or more lesion attributes;
analyzing a pathology report associated with the subject to identify one or more samples extracted from the subject, wherein each of the one or more samples has one or more sample attributes;
generating one or more mappings between the one or more lesions and the one or more samples based at least in part on correlation between one or more of the lesion attributes and one or more of the sample attributes; and
causing one or more computing devices to generate visual output about the radiology report and the pathology report simultaneously, wherein the visual output is generated based on the one or more mappings to visually emphasize a correlation between at least one of the lesions and at least one of the samples.

18. The at least one non-transitory computer-readable medium of claim 17, wherein one or more of the lesion attributes of a given lesion of the one or more lesions include a location within an anatomical structure at which the given lesion was identified.

19. The at least one non-transitory computer-readable medium of claim 18, wherein one or more of the sample attributes of a given sample of the one or more samples include a location within the anatomical structure from which the given sample was biopsied.

20. The at least one non-transitory computer-readable medium of claim 19, wherein the generating includes generating a mapping of the one or more mappings based on spatial correspondence between the location within the anatomical structure at which the lesion was identified and the location within the anatomical structure from which the sample was biopsied.
